(19) United States
(12) Patent Application Publication
(10) Pub. No.: US 2006/0205793 A1
(43) Pub. Date: Sep. 14, 2006

(54) Flupirtine preparation for the treatment of neurodegenerative diseases of the visual apparatus and diabetes mellitus

(76) Inventor: Karl-Georg Schmidt, Dresden (DE)

Correspondence Address: FROMMER LAWRENCE & HAUG, 745 FIFTH AVENUE- 10TH FL., NEW YORK, NY, 10151, US

(21) Appl. No.: 11/316,570

(22) Filed: Dec. 21, 2005

Related U.S. Application Data
(63) Continuation-in-part of application No. PCT/EP04/06738, filed on Jun. 22, 2004.

(30) Foreign Application Priority Data
Jun. 23, 2003 (DE) DE 103 28 260.2

(51) Int. Cl. A61K 31/44

(52) U.S. Cl. 514/352

(57) ABSTRACT

The invention relates to the use of flupirtine in manufacture of a pharmaceutical preparation for therapy and/or prophylaxis of diabetes mellitus, diabetic retinopathy, diabetic maculopathy, genetically related maculopathies, apoptosis of the visual apparatus and/or human glaucomas and also a combination preparation comprising flupirtine and at least one further active ingredient for therapy and/or prophylaxis of said diseases.

Preparations having a content of flupirtine are part of the prior art. Such preparations are used, for example, for the treatment of tinnitus (WO02/15907), Batten disease (WO01/39760), fibromyalgia (WO00/59487), against cell destruction due to apoptosis and necrosis (WO97/49398), impairment of the haemopoietic cell system (WO 97/17072), as an analgesic (WO97/14415), against neurodegenerative diseases (WO95/05175), against Creutzfeldt-Jakob disease (Molecule of the Month, May 2001) or as an antiphlogistic agent (DE 1795858). DE 3133519 refers generally to flupirtine medicaments.

DE 196 25 582 A1 discloses the use of flupirtine or its salts in manufacture of a medicament for prophylaxis and therapy of diseases involving a non-physiological high cell death rate. These are, on the one hand, understood to be diseases which are associated with local tissue death as the most serious consequence of local metabolic disruption, for example subsequent to mechanical, thermal, toxic or radiation damage. The death of cells can occur in different ways in the body. Whereas apoptosis (programmed cell death) constitutes an active process of cell destruction, necrosis is a consequence of non-specific damage especially to the cell membrane. DE 196 25 582 A1 additionally mentions that programmed cell death can be inhibited by means of Bcl-2, a 25 kDa membrane-based protein of about 240 amino acids. In the examples, the influence of flupirtine on the expression of Bcl-2 is investigated, and it was observed that cells which have been treated with flupirtine exhibit a markedly more intense pattern of staining with antibodies to Bcl-2. The inventors of DE 196 25 582 A1 conclude therefrom that flupirtine induces Bcl-2 expression.

DE 196 25 582 A1 does not disclose that apoptosis might have a role in the visual apparatus.

DE 43 27 516 A1 describes the use of flupirtine in neurodegenerative diseases, cerebral ischaemia and epileptic seizures.

The authors Nash and colleagues carried out studies in order to investigate whether flupirtine can prevent damage to retinal ganglion cells following ischaemia in a rat. In the model used by Nash et al., a massive increase in pressure in the eye (120 mm Hg for one hour) results in the blood supply being stopped completely, leading to an absolute lack of oxygen and nutrients. This state is referred to as ischaemia. With the exception of the so-called glaucoma attack in the case of narrow-angle glaucomas, such a massive pressure increase has no equivalent in human beings. In narrow-angle glaucomas, the drainage canals for the aqueous fluid become blocked, in which case typical pressure values are in the range 50-80 mm Hg. If such rare ischaemia in human beings remains untreated, blindness follows within 24-48 hours. The findings disclosed by Nash et al. that flupirtine might mitigate the death of ganglion cells in the rat retina cannot be transferred to human beings, because the test conditions were selected in an extreme experimental context. In no way at all, therefore, do Nash et al. speculate on use in human beings. (Nash et al., Brain Research 856 (2000), 236-239).

It has been found, surprisingly, that a preparation having a content of flupirtine can be used also in neurodegenerative diseases of the visual apparatus, such as glaucoma (cell death mechanism in the case of glaucoma), or diabetes mellitus and maculopathy, especially age-related maculopathy, but also genetically related maculopathies, retinitis pigmentosa, and also apoptosis of the visual apparatus.

An embodiment of the invention accordingly relates to a pharmaceutical preparation against neurodegenerative diseases of the visual apparatus, characterised by a content of flupirtine.

The pharmaceutical preparation according to the invention can be provided against glaucoma, diabetes mellitus and/or diabetic retinopathy.

A further embodiment of the invention relates to a pharmaceutical preparation against maculopathy, especially age-related maculopathy, diabetic maculopathy, but also genetically related maculopathies, characterised by a content of flupirtine.

A further embodiment of the invention relates to a pharmaceutical preparation against retinitis pigmentosa, characterised by a content of flupirtine.

A further embodiment of the invention relates to a pharmaceutical preparation against apoptosis of the visual apparatus, characterised by a content of flupirtine.

The pharmaceutical preparation according to the invention can be provided with a content of at least one customary carrier and/or at least one customary excipient.

The pharmaceutical preparation according to the invention can be characterised by an oral form of administration.

Furthermore, the preparation according to the invention can be characterised in that it is provided in the form of a tablet or in a capsule.

Furthermore, the preparation according to the invention can be characterised in that it is provided in a sustained-release form of administration.

Furthermore, the preparation according to the invention can be characterised in that it is provided in the form of a tablet or in a capsule having a coating that is resistant to gastric juice.

Furthermore, the preparation according to the invention can be characterised in that it is provided in the form of a drinkable solution.

Furthermore, the preparation according to the invention can be characterised in that it is present in the form of an effervescent tablet.

Furthermore, the preparation according to the invention can be characterised in that it is provided in the form of a transdermal therapeutic system, especially a patch.

Furthermore, the preparation according to the invention can be characterised in that it is provided as a solution for use in the form of drops, especially eye drops.

Furthermore, the preparation according to the invention can be characterised in that it is provided in the form of an ointment, especially an eye ointment.

Furthermore, the preparation according to the invention can be characterised in that it is provided in the form of an infusion solution.

Furthermore, the preparation according to the invention can be characterised in that it is provided in the form of a suppository.

Furthermore, the preparation according to the invention can be characterised in that it is provided in the form of a gel.

A further embodiment of the invention relates to use of flupirtine or a preparation in the treatment of neurodegenerative diseases of the visual apparatus.

Accordingly, flupirtine or a preparation according to the invention can be used in the treatment of glaucoma, diabetes mellitus and/or diabetic retinopathy.

Furthermore, a further embodiment of the invention relates to use of flupirtine or a preparation according to the invention in the treatment of maculopathy, especially age-related maculopathy, diabetic maculopathy, but also genetically related maculopathies.

A further embodiment of the invention relates to a pharmaceutical preparation against retinitis pigmentosa, characterised by a content of flupirtine.

Finally, the invention relates to use of flupirtine or a preparation according to the invention in the treatment of apoptosis of the visual apparatus.

The invention is furthermore aimed at proposing new uses of flupirtine and also combination preparations of flupirtine and at least one further active ingredient for therapy and/or prophylaxis.

In accordance with the invention, that problem is solved by the specific embodiments of the invention that are mentioned hereinbelow.

In one embodiment, the present invention accordingly relates to use of flupirtine in manufacture of a pharmaceutical preparation for therapy and/or prophylaxis of diabetes mellitus, diabetic retinopathy, diabetic maculopathy, genetically related maculopathies, apoptosis of the visual apparatus and/or human glaucomas.

It has been found to be especially advantageous to use flupirtine in prophylaxis and/or therapy of diabetes mellitus and diabetes-related diseases.

The human glaucoma can be a high-pressure or low-pressure glaucoma, especially an open-angle glaucoma.

Hitherto, no generally accepted animal has been developed for open-angle glaucomas. The so-called “ischaemia model” disclosed in Nash et al., in which all blood supply to the structures of the eye is stopped, is not suitable for comparative studies and therefore cannot be transferred to human beings. In that model, 120 mm Hg is applied for one hour, that is to say 120 mm Hg above the mean arterial pressure, which is an extremely non-physiological pressure. It results in immediate ischaemia and therefore, in the shortest time, owing to an absolute lack of oxygen and nutrients, in the destruction of many cells within the eye. For example, the corneal endothelium, the trabecular meshwork, the iris, the ciliary body, the retina, the retinal pigmented epithelium and also the choroid are affected thereby.

In contrast thereto, the internal pressure of the eye in the case of open-angle glaucomas before therapy is generally less than 30 mm Hg. In the case of normal-pressure glaucomas, which can likewise be prevented or treated by the use of flupirtine in accordance with the invention, it is even under a value of just 21 mm Hg, which is within the range of normal values of 10-21 mm Hg. Glaucomas, especially open-angle glaucomas, are not considered retinal diseases but rather are diseases of the optic nerve or optic disc, so that treatment and/or prophylaxis is directed at preservation of optic nerves, the retinal cells then being preserved secondary thereto.

It has been found to be especially advantageous to treat high-pressure and low-pressure glaucomas, especially open-angle glaucomas, with flupirtine. It was possible to arrive at an especially surprising effect in the treatment of those glaucomas when flupirtine was administered together with other medicaments in a combination preparation. In that case, blood-pressure-lowering medicaments, blood-flow-promoting medicaments, vitamin preparations, trace elements, minerals and free-radical catchers have been found to be especially advantageous. In the case of open-angle glaucomas (internal pressure of the eye before therapy usually below 30 mm Hg, in the case of normal-pressure glaucomas below 21 mm Hg, at least 30% below initial value when treated), in contrast to narrow-angle glaucomas, the drainage of aqueous fluid is not blocked so that there is no reduction in or even complete prevention of blood supply. The course of open-angle glaucomas therefore runs over years.

In the case of diabetes mellitus, diabetic retinopathy in the case of retinitis pigmentosa and also macular degeneration, the internal pressure of the eye is usually normal (below 21 mm Hg) so that those diseases do not arise as a result of massively increased internal pressure of the eye. Instead, a disruption in sugar and/or insulin metabolism is the basis for diabetes mellitus and diabetic retinopathy. Macular degenerations are a functional impairment of the retinal pigmented epithelium, in some cases together with formation of new vessels (neovascularisation), whereas in the case of retinitis pigmentosa and macular degeneration in the case of retinitis pigmentosa the cause is usually genetically related destruction of the retinal pigmented epithelium. The course of those diseases, too, runs over years.

Furthermore, the pharmaceutical preparation in the use according to the invention comprises at least one customary carrier and/or at least one customary excipient.

The pharmaceutical preparation can, especially, be administered orally. By that means, forms of administration that are unpleasant for the patient, such as injection or direct treatment of the eye, can be avoided effectively.

In a preferred embodiment, the pharmaceutical preparation is present in the form of a tablet or in a capsule.

In addition, preference is given to the pharmaceutical preparation being in the form of a sustained-release form of administration. Sustained-release forms of administration have the particular advantage that release of the active ingredient in the body of the patient takes place continuously, spread out over a long period of time. It is accordingly possible for an initial “overdose”, which then drops off markedly however, to be effectively avoided.

Furthermore, the tablet or capsule can have a coating that is resistant to gastric juice.

The pharmaceutical preparation can also be present in the form of a drinkable solution.

Furthermore, the pharmaceutical preparation can be present in the form of an effervescent tablet.

In addition, preference is given to the pharmaceutical preparation being present in the form of a transdermal therapeutic system, especially a patch, a solution for use in the form of drops, an ointment, especially an eye ointment, an infusion solution, suppositories, a gel and/or a medicament carrier, especially an ocusert.

Medicament carriers (ocuserts), which are placed directly onto the conjunctiva and which constantly release flupirtine and also, as the case may be, a further active ingredient constantly, have been found to be valuable in practice.

The invention relates also to a combination preparation comprising flupirtine and at least one further active ingredient for therapy and/or prophylaxis of diabetes mellitus, diabetic retinopathy, diabetic maculopathy, genetically related maculopathies, apoptosis of the visual apparatus and/or human glaucomas.

Finally, flupirtine and at least one further active ingredient can be used in manufacture of a combination preparation which is used in therapy and/or prophylaxis of diabetes mellitus, diabetic retinopathy, diabetic maculopathy, genetically related maculopathies, apoptosis of the visual apparatus and/or human glaucomas.

The invention will be explained in greater detail by means of Examples.

### EXAMPLE 1

### Glaucoma

Katadolon capsules were used.
Constituents: Flupirtine maleate
   Calcium hydrogen phosphate
   Copovidone
   Magnesium stearate
   Colloidal silica
   Gelatin
   Colourings: E171, E172
   Sodium dodecyl sulfate

The treatment was carried out on a male patient, who for more than 13 years had been suffering from a progressing glaucoma, who was being treated conservatively, and who was provided with filtration surgery.

The patient was treated for 6 years with the mentioned preparation with continuation of the conservative therapy. In that period, no deterioration in findings occurred. The treatment accordingly protected against progression of the glaucoma.

### EXAMPLE 2

### Glaucoma

The above-mentioned capsules were used.

The treatment was carried out on a female patient, who for more than 15 years had been suffering from a progressing glaucoma and who was being treated conservatively.

The patient was treated for 5 years with the mentioned preparation with continuation of the conservative therapy. In that period, no deterioration in findings occurred. The treatment accordingly protected against progression of the glaucoma.

### EXAMPLE 3

### Progressing Diabetes Mellitus with Progressing Diabetic Retinopathy

The above-mentioned capsules were used.

The treatment was carried out on a male patient, who for more than 26 years had been suffering from progressing diabetes mellitus with progressing diabetic retinopathy, who was being treated conservatively and who was provided with repeated laser treatments of the retina.

The patient was treated for 4 years with the mentioned preparation with continuation of the conservative therapy. In that period, a substantial stabilisation in findings occurred. The treatment accordingly protected against progression of the diabetes mellitus and of the diabetic retinopathy.

The patient was then treated with a combination preparation comprising flupirtine and vitamin C for two years with continuation of the conservative therapy. In that period, a marked improvement in findings occurred. The combination therapy accordingly not only protects against progression of the diabetes mellitus and of the diabetic retinopathy but can even improve them markedly.

### EXAMPLE 4

### Progressing Diabetes Mellitus with Progressing Diabetic Retinopathy

The above-mentioned capsules were used.

The treatment was carried out on a male patient, who for more than 22 years had been suffering from progressing diabetes mellitus with progressing diabetic retinopathy, who was being treated conservatively and who was provided with repeated laser treatments of the central and peripheral retina.

The patient was treated for 7 years with the mentioned preparation with continuation of the conservative therapy. In that period, a substantial stabilisation in findings occurred. The treatment accordingly protected against progression of the diabetes mellitus and of the diabetic retinopathy.

The patient was then treated with a combination preparation comprising flupirtine and vitamin C for two years with continuation of the conservative therapy. In that period, a marked improvement in findings occurred. The combination therapy accordingly not only protects against progression of the diabetes mellitus and of the diabetic retinopathy but can even improve them markedly.

### EXAMPLE 5

### Age-Related Maculopathy

The above-mentioned capsules were used.

The treatment was carried out on a female patient, who for more than 6 years had been suffering from a progressing age-related maculopathy and had been provided with laser treatment of the macula.

The patient was treated for 6.5 years with the mentioned preparation. In that period, a substantial stabilisation in findings occurred. The treatment accordingly protected against progression of the age-related maculopathy.

### EXAMPLE 6

### Age-Related Maculopathy

The above-mentioned capsules were used.

The treatment was carried out on a female patient, who for more than 8 years had been suffering from a progressing age-related maculopathy. The patient was treated for 4 years with the mentioned preparation. In that period, a substantial stabilisation in findings occurred. The treatment accordingly protected against progression of the age-related maculopathy.

### EXAMPLE 7

### Retinitis Pigmentosa with Progressing Maculopathy

The above-mentioned capsules were used.

The treatment was carried out on a female patient, who for more than 14 years had been suffering from a retinitis pigmentosa with progressing maculopathy.

The patient was treated for 4 years with the mentioned preparation. In that period, a substantial stabilisation in findings occurred. The treatment accordingly protected against progression of the retinitis pigmentosa and the retinitis pigmentosa-related maculopathy.

### EXAMPLE 8

### Retinitis Pigmentosa

The above-mentioned capsules were used.

The treatment was carried out on a male patient, who for more than 19 years had been suffering from a retinitis pigmentosa.

The patient was treated for 7 years with the mentioned preparation. In that period, a substantial stabilisation in findings occurred. The treatment accordingly protected against progression of the retinitis pigmentosa.

1) Use of flupirtine in manufacture of a pharmaceutical preparation for therapy and/or prophylaxis of diabetes mellitus, diabetic retinopathy, diabetic maculopathy, genetically related maculopathies, apoptosis of the visual apparatus and/or human glaucomas.

2) Use according to claim 1, wherein the human glaucoma is a high-pressure or low-pressure glaucoma, especially an open-angle glaucoma.

3) Use according to claim 1 or 2, wherein the pharmaceutical preparation has at least one customary carrier and/or at least one customary excipient.

4) Use according to one of the preceding claims, wherein the pharmaceutical preparation is administered orally.

5) Use according to one of the preceding claims, wherein the pharmaceutical preparation is present in the form of a tablet or in a capsule.

6) Use according to one of the preceding claims, wherein the pharmaceutical preparation is present in the form of a sustained-release form of administration.

7) Use according to claim 5 or 6, wherein the tablet or capsule has a coating that is resistant to gastric juice.

8) Use according to one of claims 1 to 4, wherein the pharmaceutical preparation is present in the form of a drinkable solution.

9) Use according to claim 8, wherein the pharmaceutical preparation is present in the form of an effervescent tablet.

10) Use according to one of claims 1 to 3, wherein the pharmaceutical preparation is present in the form of a transdermal therapeutic system, especially a patch, a solution for use in the form of drops, especially eye drops, an ointment, especially an eye ointment, an infusion solution, suppositories, a gel and/or a medicament carrier, especially an ocusert.

11) Combination preparation comprising flupirtine and at least one further active ingredient for therapy and/or prophylaxis of diabetes mellitus, diabetic retinopathy, diabetic maculopathy, genetically related maculopathies, apoptosis of the visual apparatus and/or human glaucomas.

12) Use of flupirtine and at least one further active ingredient in manufacture of a combination preparation for therapy and/or prophylaxis of diabetes mellitus, diabetic retinopathy, diabetic maculopathy, genetically related maculopathies, apoptosis of the visual apparatus and/or human glaucomas.
